(19) United States
(12) Patent Application Publication
(10) Pub. No.: US 2022/0240609 A1
(43) Pub. Date: Aug. 4, 2022

(54) Far-UVC Glove for Anti-Virus Protection

(71) Applicants: Michele Marie Day, Youngsville, LA (US); Alan Cook Day, Youngsville, LA (US)

(72) Inventors: Michele Marie Day, Youngsville, LA (US); Alan Cook Day, Youngsville, LA (US)

(21) Appl. No.: 17/163,361

(22) Filed: Jan. 30, 2021

(51) Int. Cl. A41D 19/00

(52) U.S. Cl. CPC A41D 19/0024; A41D 2500/50

(57) ABSTRACT

Wearable Far-Ultra-Violet C (Far-UVC) finger thimble article with components thereof and other accessories is provided to protect wearer from intrusion of viruses and micro-organisms. The finger thimble article is integrated with Far-UVC diodes on a finger thimble that project wavelengths of about 222 nanometers to protect against viruses. The finger thimble article would be a type of finger thimble covering with Far-UVC diodes attached on the fingertips projecting sterilizing rays on surfaces at a safe distance protecting skin and eyes.

### BACKGROUND

With the pandemic of COVID19 and other types of viruses, sterilization while traveling or in crowded venues is key in maintaining health. Far-UVC light can be used for disinfecting, sterilizing, and destroying harmful micro-organisms on surfaces and in the air. In addition, parasitic insects have preyed on humans and animals causing diseases and even death. Application of Far-UVC lights article on gloves, may be beneficial to eliminating viruses and insects before infecting humans.

Far-UVC lights are used to sterilize surfaces and when combined with wearable gloves may prove beneficial in reducing the spread of viruses within crowds at hospitals, emergency rooms, construction sights, athletic stadiums, festivals, carnivals, schools, colleges, churches, work environments, airports, malls, cities, or any venue with crowds of people. Far-UVC lights could be used in camping environments, farming, and any outdoor activity for humans and animals to remain safe from parasitic insects.

Far-UVC combined with wearable gloves could potentially kill viruses living on money, cellphones or other objects used for handling. Scientific studies have shown that viruses can live on some surfaces for up to 28 days and on skin for 11 days.

Far-UVC lights are currently found within wand applications, air filtration, and indoor lighting but have yet to be combined with wearable gloves.

Therefore, a need exists for novel application of positioning Far-UVC lights within wearable gloves to eliminate viruses or other micro-organisms before infecting a human.

### REFERENCES CITED

With regard to the reference of the patent, Wang et al. (US 2005/0186258), differences remain in the distinction of the Wearable Far-UVC glove invention where the illumination of a shorter wavelength disseminates off the glove verses a UV packaging or encasement for gloves utilized for killing viruses as designed by the Wang patent. The Wearable Far-UVC with integration in hand coverings allows for this invention with integration of Far-UVC photons to emit externally from the glove into the air or surfaces at 222 nanometers to kill viruses. This wavelength allows for the scientifically studied safety of Far-UVC rays near skin and eyes verses UV rays at a higher level mentioned in Wang's patent which is dangerous to skin. The UV mentioned in the Wang patent likely ranges from at 315-400 nanometers which makes it dangerous to exposed skin. This invention with the implementation of Wearable Far-UVC at approximately 222 nanometers emits externally from the hand coverings versus a packaging or encasement for the glove with a UV light as specified by the Wang patent. The difference remains that this invention designed with Far-UVC is illuminated externally from the glove fingertips versus a glove placed in a UV encasement specified by Wang's design. Far-UVC at a low level maintains scientifically safe standards to be on or near the skin and eyes while killing viruses in the air. In conclusion, the Wearable Far-UVC invention differs from the Wang patent referenced.

With regard to the reference of the patent, Modak and Sampath (U.S. Pat. No. 5,133,090A), differences remain in the distinction of the Wearable Far-UVC glove invention where the illumination of approximately 222 nanometer wavelength article disseminates off the glove verses the Modak and Sampath patent consisting of an inner coating antibacterial agent within the glove.

With regard to the reference of the patent, [symbol] (KR200390225Y1), differences remain in the distinction of the Wearable Far-UVC invention where the illumination of approximately 222 nanometer wavelength article disseminates off the glove verses the patent by [symbol] whereas the glove is coated with photocatalyst (titanium dioxide, Tio₂) to cause oxidative chemical reactions with ultraviolet rays emitted from an external light source to reduce various pathogens, bacteria and contaminants.

With regard to the reference of the patent, Hamasaki (US20200146375A1), differences remain in the distinction of the Wearable Far-UVC glove invention where the illumination of approximately 222 nanometer wavelength article disseminates off the glove verses the patent by Hamasaki where the glove protects from a UV light which disseminates at a higher wavelength. The Hamasaki patent design is to be used during the gel manicure process, for example at a nail salon. The gloves allow exposure of the fingernails to enable a nail technician to perform treatments while simultaneously protecting the guest's hands from UV rays.

With regard to the reference of the patent, Teital (US20140318564), differences remain in the distinction of the Wearable Far-UVC invention where the illumination of approximately 222 nanometer wavelength article disseminates off the glove verses the patent by Teital where the glove or sock protects from a UV light which disseminates at a higher wavelength. The Teital patent design is to be used during the manicure process, for example at a nail salon. The gloves allow exposure of the fingernails to enable a nail technician to perform treatments while simultaneously protecting the guest's hands from UV rays.

### BRIEF SUMMARY OF THE INVENTION

The present invention comprises a novel wearable Far-UVC light article with wearable gloves. A novel design of the Far-UVC lights on gloves emitting a wavelength of approximate 222 nanometers will allow the light to be activated when needed in crowds. The Far-UVC light is illuminated on the fingertips of the exterior of the gloves to prevent micro-organisms from landing on the skin with its sterilization properties. Far-UVC lights positioned on the outside of a glove could prevent the virus from infecting humans and could potentially clean viruses off surfaces. Far-UVC lights will be affixed in a position on the fingertips to sterilize and prevent viruses from infection. Far-UVC at approximately 222 nanometers for wearable gloves is safe to human cells due to the lower wavelength.

### BRIEF DESCRIPTION OF THE DRAWING

Some embodiments of the present invention are illustrated as an example and are not limited by the figures of the accompanying drawings, in which like references may indicate similar elements and in which:

FIG. 1—FIG. 1 depicts an embodiment of a glove with Far-UVC photon articles affixed to the fingertips of the glove to potentially prevent the virus from attaching to the human hand as well as sterilizing surfaces touched with the Far-UVC wavelengths.

FIG. 2—FIG. 2 depicts an exploded perspective view of one example of the Far-UVC article placements on a wearable glove according to various embodiments of the present invention.

FIG. 3—FIG. 3 depicts an embodiment of a glove with the Far-UVC wavelengths of approximately 222 nanometers emanating off the Far-UVC photon article affixed to the fingertip of the glove.

### DETAILED DESCRIPTION OF THE INVENTION

The terminology used herein is for the purpose of describing particular embodiments only and is not intended to be limiting of the invention. As used herein, the term “and/or” includes any and all combinations of one or more of the associated listed items. As used herein, the singular forms “a”, “an” and “the” are intended to use the plural forms as well as the singular forms, unless the context clearly indicates otherwise. It will be further understood that the terms “comprise” and/or “comprising” when used in this specification, specify the presence of stated features, steps, operations, elements, and/or components, but do not preclude the presence or addition of one or more of the features, steps, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formerly sense unless expressly so defined herein.

In describing the invention, it will be understood that a number of techniques and steps are disclosed. Each of these has individual benefit and each can be used in conjunction with one or more, or in some cases all, of the other disclosed techniques. Accordingly, for the sake of brevity, this description will refrain from repeating every possibly combination of the individual steps in an unnecessary fashion. Nevertheless, the specification and claims should be read with the understanding that such combinations are entirely within the scope of the invention and the claims.

Novel Wearable Far-UVC articles are positioned on gloves and attire to repel micro-organisms from attaching to humans. In the following description, for purposes of explanation, numerous specific details are set forth in order to provide a thorough understanding of the present invention. It will be evident, however, to one skilled in the art that the present invention may be practiced without these specific details.

The present disclosure is to be considered as an exemplification of the invention and is not intended to limit the invention to the specific embodiment illustrated by the figures or description below.

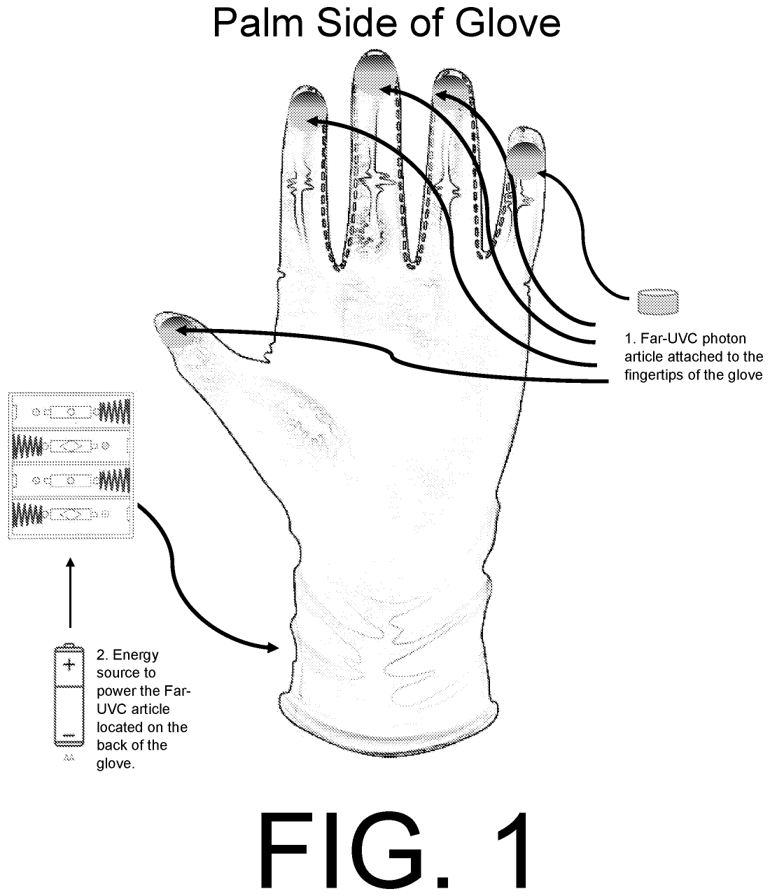

The present invention will now be described by referencing the appended figures representing preferred embodiments. FIG. 1 depicts the palm side view of the glove with the Far-UVC photon articles affixed to the fingertips.

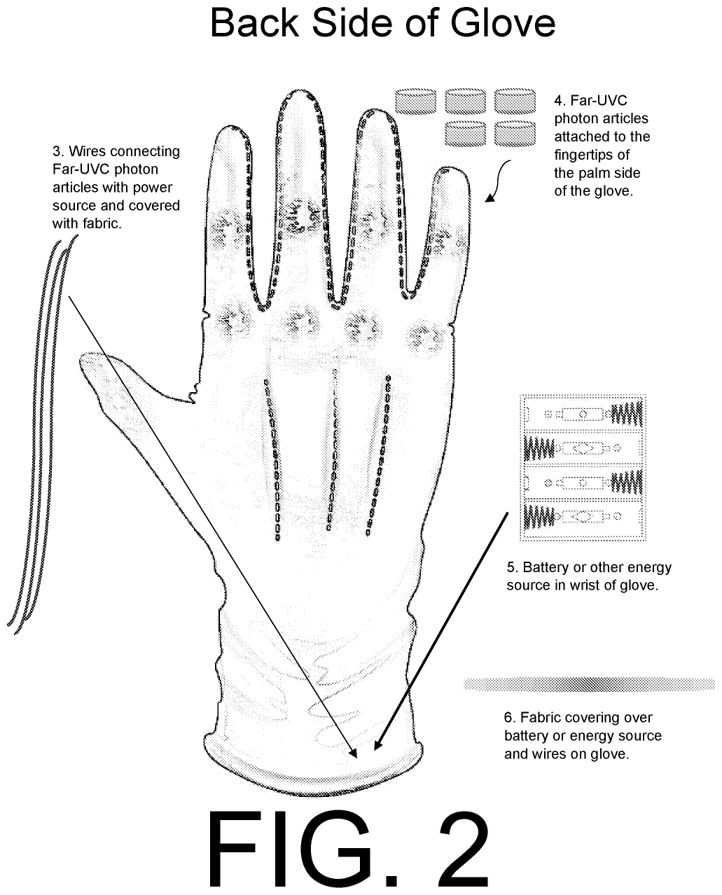

In FIG. 2 the elements of the invention represented depicts an exploded perspective view of the elements that may comprise a photon articles, energy source, and wires affixed onto the wrist portion of the glove.

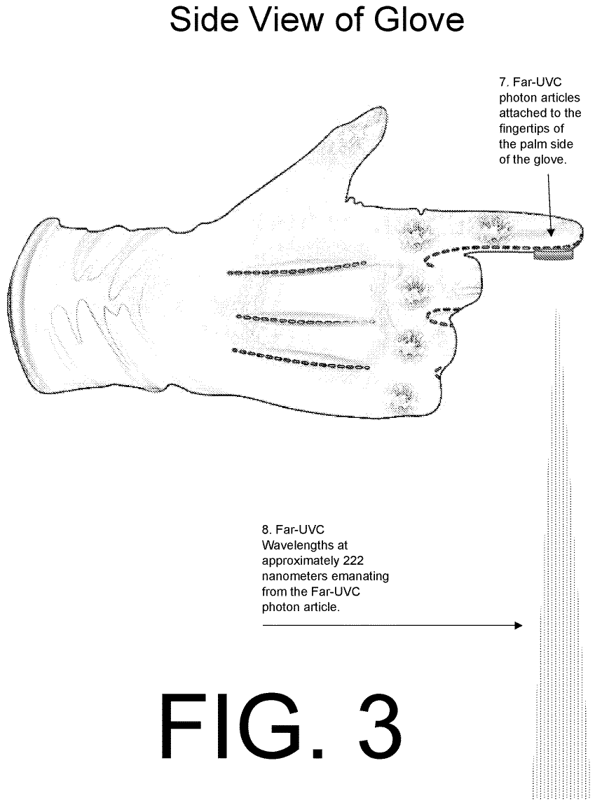

In FIG. 3 the elements of the invention are represented as a side view of the glove with the Far-UVC wavelengths at approximately 222 nanometers emanating from the Far-UVC photon article.

While preferred materials for elements have been described, the device is not limited by these materials. Fabric, plastics, latex, rubber, foam, wood, metal alloys, aluminum, and other materials may comprise some or all of the elements of the positioning devices and apparatuses in various embodiments of the present invention.

Although the present invention has been illustrated and described herein with reference to preferred embodiments and specific examples thereof, it will be readily apparent to those of ordinary skill in the art that other embodiments and examples perform similar functions and/or achieve like results. All such equivalent embodiments and examples are within the spirit and scope of the present invention, are contemplated thereby, and are intended to be covered by the aforementioned claims.

1. Far-UVC Finger Thimble article for anti-virus protection comprising:
a finger thimble pitted cup covering a fingertip;
a Far-UVC holder is positioned on the fingertip pitted cup surface substantially adjacent to the outer perimeter edge;
a Far-UVC diode emitting a wavelength of approximately 222 nm is secured to the Far-UVC holder substantially downward between about 90 degrees to about 120 degrees perpendicular to a horizontal axis of the ledge.

2. A Far-UVC Finger Thimble of claim 1 comprising a power source of one of a rechargeable battery, solar power, electromagnetism, or acoustic sound.

3. A Far-UVC Finger Thimble of claim 1 comprising the thimble including one of fabric, metal, or plastic.
